(19) United States
(12) Patent Application Publication
(10) Pub. No.: US 2007/0032852 A1
(43) Pub. Date: Feb. 8, 2007

(54) Methods and Apparatus for Treatment of Aneurysms Adjacent to Branch Arteries

(75) Inventors: James Machek, Windsor, CA (US); Scott Doig, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/464,591

(22) Filed: Aug. 15, 2006

Related U.S. Application Data
(63) Continuation-in-part of application No. 10/423,297, filed on Apr. 25, 2003.
(60) Provisional application No. 60/710,798, filed on Aug. 23, 2005.

(51) Int. Cl. A61F 2/06; A61F 2/90

(52) U.S. Cl. 623/1.13; 623/1.35

(57) ABSTRACT

A stent graft includes a pair of generally opposed windows alignable with the superior mesentery artery, the celiac trunk and separated by graft material. A stent is provided spanning the window and in contact with the adjacent graft material and the stent presses the adjacent graft material into engagement with the adjacent wall of the flow lumen. A branch graft connection into the renal arteries may also be provided.

### RELATED APPLICATIONS

This application claims the benefit of U.S. Provisional Application No. 60/710,798 filed Aug. 23, 2005 and is a Continuation-in-Part of U.S. application Ser. No. 10/423,297, filed Apr. 25, 2003.

### FIELD OF THE INVENTION

The field of the invention is the treatment of vascular abnormalities. More particularly, the field of the invention is the treatment of vascular abnormalities by placing an excluding device in a blood vessel to exclude or bypass an abnormality, including placing such an excluding device in an area near one or more branch vessels so as to bypass the abnormality, but not occlude the branch vessel.

### BACKGROUND OF THE INVENTION

“Aortic aneurysm” is the term used to describe a vascular abnormality condition where a segment of the aorta is dilated to a diameter greater than its original diameter. Aneurysms can occur in virtually any region of the vasculature including the aorta in the abdominal and thoracic regions. Aortic aneurysms are caused by hardening of the arteries (atherosclerosis), high blood pressure (hypertension), genetic disposition such as Marfan's Syndrome, trauma or less common disorders. Atherosclerosis is the most common cause.

Where dilation of the aorta meets or exceeds 50% of the original aortic diameter, i.e., where the diameter of the aorta is 150% of the original or expected diameter, intervention generally is deemed necessary. Without intervention, the aneurysm may continue to expand, leading to the possibility of tearing or rupture of the aorta and death. Intervention includes techniques such as replacement of the aorta with a synthetic lumen which is sewn to the two ends of the still viable aorta after the aneurysmal portion has been opened or surgically removed, or, less invasively, by the endovascular placement of an exclusion device such as a stent graft across the aneurysmal site. The stent graft is a tubular member designed to provide a conduit enabling blood flow through the aorta without allowing the systemic pressure of the blood to further stretch the aneurysm. For this intervention to be successful, the stent graft must span the weakened blood vessel wall so that the stent grafts' opposed ends engage and seal against healthy blood vessel tissue on the proximal and distal sides of the aneurysm.

A stent graft includes a stent (framework) portion which provides physical support of the stent graft in a tubular configuration once deployed at a vascular location, and a graft portion, comprising an excluding material, which is sewn or otherwise attached to the stent portion and which provides a relatively fluid-tight conduit for blood flow through the stent graft and past the aneurysm site. Placement of a stent graft can be performed without a chest incision, by using specialized catheters that are introduced through arteries usually at a location in a leg adjacent to the groin.

The aorta has numerous arterial branches. For example, the descending aorta includes the superior mesentery artery, the celiac trunk and the renal arteries. The proximity of an aneurysm to a branch artery may limit the use of an excluding device such as a tubular stent graft, as the main body or ends of the tubular stent graft may occlude or block the branch arteries due to the positioning of the stent graft at the location of healthy artery wall. Alternatively, there may be an inadequate length of healthy tissue for the stent graft to seal against in the area between the aneurysmal region of the artery and the location of the branch arteries. In this case, even if the stent graft initially is located without blocking a branch artery, there still is a risk of migration of the exclusion device to a position where it may partially or fully block a branch artery. Additionally, where multiple branch arteries are present adjacent to the aneurysm, the ability to position a stent graft so as not to occlude any of the branch arteries may be problematic. Furthermore, the aneurysm may implicate the aortic wall tissue adjacent to the branch arteries, such as the renal arteries, such that the aorta is dilated at the renal arteries, and the stent graft must extend over the renal arteries to seal against healthy aorta wall tissue. Therefore, there is a desire in the art to achieve a greater success of aneurysm repair and healing, and in particular, mechanisms and methods to enable stent grafting or the placement of other exclusion devices adjacent to branch vessels in aneurysmal locations.

### SUMMARY OF THE INVENTION

Embodiments according to the present invention address aneurysm repair and positional stability of a device used for aneurysm repair. Specifically, embodiments according to the present invention provide methods and apparatus for use in the treatment of aneurysms located near branch vessels, using windowed stent grafts. Thus, in one embodiment according to the invention there is provided an exclusion device useful for implantation in an aneurysmal site in a blood vessel having a branch vessel near the aneurysmal site comprising: a main body having at least one pair of apertures therein alignable with branch arteries, and one or more windows therein, the windows being sized and arraigned such that upon deployment of the stent graft in a body flow lumen, the windows provide for flow of fluids from a main body lumen into a branch lumen without impeding the flow into the branch lumen. Simultaneously, the portion of the exclusion device framing or bordering the windows provides additional anchoring of the exclusion device in place in the aorta by being biased into contact with the aorta wall adjacent to and between the branch artery locations. In one aspect, the window portion(s) are configured as flaps of graft material, supported on a stent or stents in a stent frame, separated in a circumferential direction by scalloped open regions which form the windows. In another embodiment, the window portion(s) are provided as fully surrounded apertures through the graft portion of the exclusion device. The windows are provided to align, when the exclusion device is deployed in an aneurysmal aorta, with branch arteries such as those adjacent to the renal arteries, with a relatively large level of positional tolerance as compared to the position of the branch arteries, to enable relatively simple deployment of the exclusion device where the exclusion device must be deployed across multiple branch vessels, and to provide rotational and longitudinal support to help prevent migration of the exclusion device from the deployed position in the aorta. The apertures provided for alignment with additional branch arteries may include a spanning device(s), such as a secondary tubular structure or grommet, which extends outwardly from the body of the exclusion device and into sealing engagement with the adjacent branch artery. In one aspect, these adjacent branch arteries are the renal arteries, and the exclusion device, adjacent to the aperture(s) is not in direct contact with the aorta wall, such that the secondary tubular structure(s) span a gap between the body of the exclusion device and the renal artery(s) to provide a sealed passage for flow of blood through the exclusion device and secondary tubular structures to provide flow through the renal artery(s) without allowing such flow to leak into the sealed off aneurysmal region.

In a further aspect, the exclusion device is configured as a stent graft, having a stent frame and a graft material formed thereover and attached thereto. In this configuration, at least one pair of framed apertures are provided, which are alignable with, when the stent graft is deployed, branch lumens from a main flow lumen, and at least one window extending through the graft portion for alignment with an additional branch vessel. In an additional aspect, at least one grommet is provided within at least one of the framed apertures, such grommet being provided with a passage therethrough for flow of fluid from the main flow lumen to the branch lumen which is aligned with a branch lumen, such that the grommet is secured within the aperture and extends into the branch flow lumen and provides a flow conduit therethrough. Additionally the window may be configured as a slot extending inwardly from one end of the stent graft, having a stent of the stent frame such that at least one flap of graft material extends adjacent the slot and a stent is secured to the flap of graft material.

In yet a further aspect, according to the invention, a deployment device, such as a catheter, within which the stent graft is held prior to deployment thereof into a flow lumen adjacent to one or more branch lumens extending from the flow lumen. The stent graft is deployed from the catheter at an aneurysmal location, such that at least one aperture thereof aligns with a branch artery, and at least one window extends through the stent graft and is aligned with an additional branch artery adjacent to the aneurysm location.

### BRIEF DESCRIPTION OF THE DRAWINGS

A more particular description of the embodiments may be had by reference to the embodiments according to the invention described in the present specification and illustrated in the appended drawings. It is to be noted, however, that the specification and appended drawings illustrate only certain embodiments according to this invention and are, therefore, not to be considered to be limiting of its scope.

FIG. 1 is a cross sectional schematic view of an abdominal aortic aneurysm;

FIG. 2 is the schematic view of the abdominal aortic aneurysm of FIG. 1, wherein the aneurysm has been excluded by a stent graft according to the present invention;

FIG. 3 is a partial perspective view of a portion of the exclusion device shown in FIG. 2;

FIGS. 3A and 3B are alternate embodiments of the structure shown in FIG. 3;

FIG. 4 is a partial perspective view of an alternative construction of a portion of the exclusion device shown in FIG. 2;

FIG. 5 is a sectional view of a portion of FIG. 2, showing the interconnection of the body and a renal extension therefore of the exclusion device of FIG. 2;

FIG. 6 is a perspective view of a single stent useful for incorporation into the exclusion device of FIG. 2;

FIG. 7 is a side view of a stent (framework) portion useful for incorporation into the exclusion device of FIG. 2;

FIG. 8 is a partial perspective view of an additional alternative construction of a portion of the exclusion device shown in FIG. 2;

FIG. 9 is a partial perspective view of a delivery device for deploying the exclusion device of FIG. 2;

FIG. 10 is schematic view of a portion of the delivery device of FIG. 9 positioned for beginning deployment of the exclusion device of FIG. 2 across the abdominal aortic aneurysm;

FIG. 11 is a schematic view of the delivery device of FIG. 9 beginning deployment of the exclusion device for spanning the abdominal aortic aneurysm;

FIG. 12 is a schematic view of a portion of the exclusion device deployed to span an abdominal aortic aneurysm;

FIG. 13 is a schematic view of an additional delivery device positioned to deploy a leg of the exclusion device of FIG. 12 into the previously deployed portion of the exclusion device;

FIG. 14 is a schematic view of the leg of the exclusion device deployed adjacent to the abdominal aortic aneurysm;

FIG. 15 is a schematic view of the beginning of deployment of one of the renal extensions into the body of the exclusion device of FIG. 14;

FIG. 16 is a partial schematic view of the abdominal aortic aneurysm of FIG. 1 showing the deployment position of an additional deployment device for deploying the renal extensions;

FIG. 17 is a partial schematic view of the abdominal aortic aneurysm of FIG. 1 showing a renal extension deployed to span the gap between the body of the extension device and the adjacent aorta wall.

### DETAILED DESCRIPTION

Reference now will be made to details of exemplary embodiments according to the invention. It is to be understood that the described embodiments are not intended to limit the invention solely and specifically to only these embodiments.

Methods and apparatus for stabilizing and treating an aneurysm include deploying an exclusion device, such as a stent graft, in the flow lumen of a blood vessel to span the aneurysmal location and seal off the aneurysmal location of the blood vessel from further blood flow while acting as a conduit to direct blood flow past the aneurysmal site. In the case of an aneurysm near a branch artery, methods and apparatus for treatment include positioning an endovascular stent graft in the aneurysmal site, where the stent graft includes a body with a pair of opposed apertures, where separate individual inserts are disposed within each aperture to extend sealingly into the exclusion device and sealingly into a branch artery, and at least one additional window extending through the graft portion of the stent graft to allow the stent graft to extend around additional branch arteries without blocking them off from the main flow lumen.

Each of the apertures of the main body of the stent graft is alignable with, and includes mechanisms extendable into, a branch artery to aid in maintaining alignment with the branch artery and for spanning any gap between the stent graft and the adjacent aorta wall. The stent graft excludes the weakened vessel wall at the aneurysmal site from further exposure to blood flowing through the aorta, but, as a result of the aperture, allows blood to flow from the aorta to the branch artery(ies), even where the main body of the stent graft extends past the branch artery(ies). Inserts are provided to fit sealingly into each aperture and further extend sealingly into the branch vessels, thereby preventing leakage of blood from the branch arteries into the region between the stent graft and the weakened blood vessel wall at the aneurysmal location.

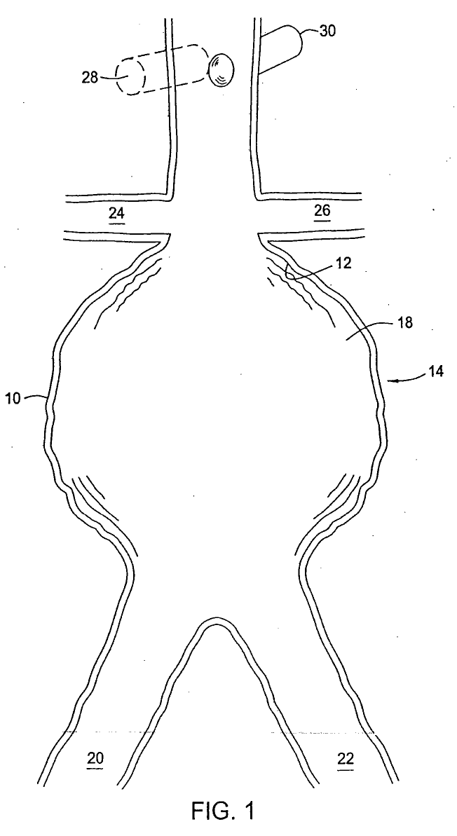

Referring initially to FIG. 1, there is shown an aneurysm of the thoracic aorta 10, such that the aorta 10 is enlarged at an aneurysmal location 14 at which the aorta wall 12 is distended and stretched. The distended and stretched aneurysmal location 14 forms an aneurysmal bulge or sac 18. If left untreated, the aneurysmal wall 12 may continue to deteriorate, weaken, and eventually tear or burst. In the aorta 10 shown in FIG. 1, the aneurysmal sac 18 is located adjacent to, and on the upstream (blood flow direction) side of, the branching of the aorta 10 into the right iliac artery 20 and left iliac artery 22, and adjacent to the renal arteries 24, 26, and ends intermediate of the renal arteries 24, 26 and the mesentery artery 30 and celiac trunk 28 (shown in phantom) such that the aorta 10 is dilated at the renal artery 24, 26 location. Thus, to exclude the aneurysmal sac 18, the excluding device must span over the renal arteries, and, seal against the aorta wall 12 at a location upstream of the renal arteries 24, 26.

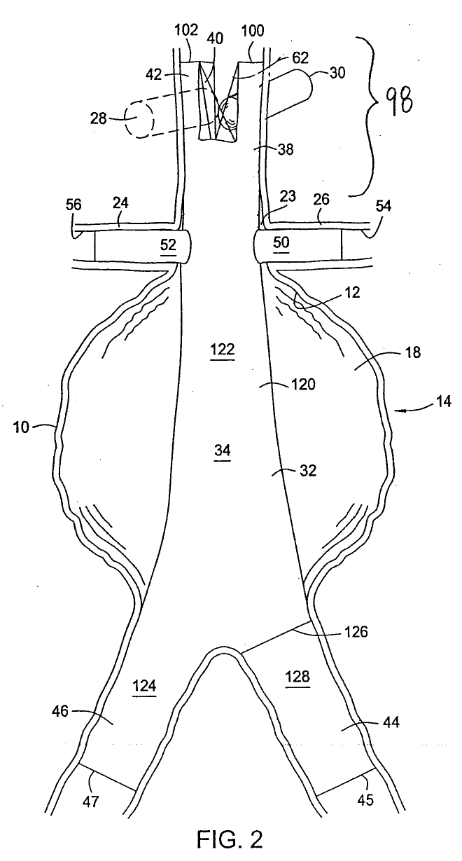

Referring now to FIG. 2, a stent graft 32 is shown deployed in the aorta 10 to exclude the aneurysmal sac 18 and sealingly engage against the aorta wall 12 at locations on either side of the aneurysmal sac 18. Stent graft 32 generally includes a body 34 formed of graft material 38 and a stent frame (work) 40 (of which only a single stent 62 may be seen in FIG. 2) as will be further described herein, and includes a first end 42 deployed upstream, from a blood flow perspective, from the renal arteries 24, 26, and at its opposite end, bifurcated right and left iliac legs 44, 46 terminating in open left and right ends 45, 47 respectively. Stent graft 32, when deployed, sealingly engages against the inner walls of the iliac arteries 20, 22 by engagement of the stent graft 32 against the artery walls adjacent to the ends 45, 47 of the legs 44, 46 thereof, and extends therefrom to a position upstream, in a blood flow direction, to a location adjacent to the superior mesentery artery 30 and the celiac trunk 20 and seals against the artery wall 12 at a position located between the position of the intersection of the renal arteries 24, 26 with the aorta 10 and that of the intersection of the superior mesentery artery 30 and the celiac trunk 20 with the aorta 10. Thus, the stent graft 32 provides exclusion of the aneurysmal sac 18 while providing a blood flow bypass of the aneurysmal sac 18 through the hollow interior of the stent graft 30. Additionally, as the aorta 10 is dilated at the renal arteries 24, 26, there is a gap 23 between the stent graft 32 and the aorta wall 12 at these locations. To enable blood flow from the aorta 10 into the renal arteries 24, 26, and simultaneously seal off the adjacent aneurysmal sac 18, the stent graft 32 may also include a pair of generally opposed renal extensions 50, 52, which extend across any gap between the stent graft 32 and the aorta wall 12 and into sealing engagement against the inner walls 54, 56 of the renal arteries 24, 26 while allowing fluid flow from the hollow interior of the body 34 therethrough. Additionally, the first end 42 of the stent graft 32 is configured to extend, in an upstream direction, past the location of the superior mesentery artery 30 and the celiac trunk 20 without occluding or blocking the openings thereof from the aorta 10 as will be further described herein.

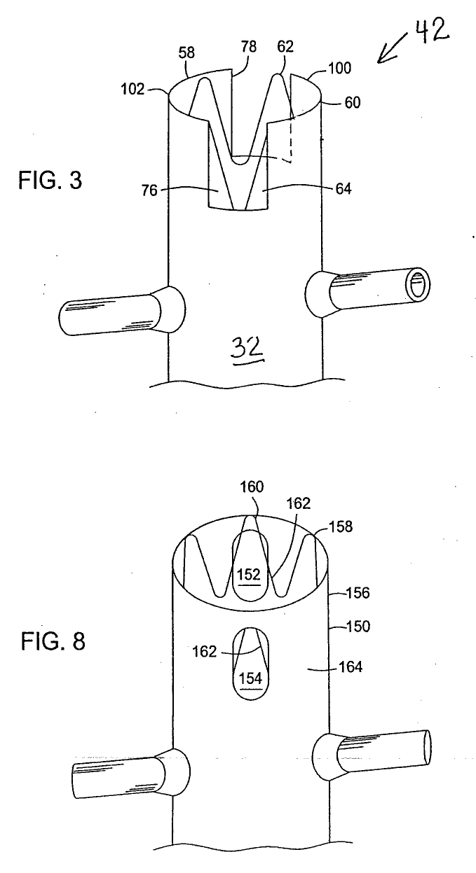

Referring now to FIG. 3, the structure and arrangement of the first end 42 of the stent graft 32 for engagement against the aorta wall 12 adjacent and between the intersection of the aorta 10 with the superior mesentery artery 30 and the celiac trunk 20 is shown. Specifically, the first end 42 of the stent graft 32 is shown in FIG. 3 in a perspective view, wherein a stent 62 forming a portion of a stent frame 64 (only a portion thereof shown in FIG. 3) is shown received within the open first end 42 of stent graft 32, and the first end 42 is also modified from a generally right cylindrical envelope of stent material 38 to include two windows 76, 78 which are disposed between two flaps 58, 60 which frame each of the windows 76, 78 on three sides thereof where the windows have a generally rectangular profile as shown in FIG. 3. Each of windows 76, 78 is preferably formed by cutting diametrically opposed, in reference to the tubular graft material 38, slots inwardly of the first end 42 of the stent graft 32 to form windows 76, 78 such that flaps 58, 60 of graft material 38 remain between the windows 76, 78. The cut edge of the slots forming windows 76, 78 are heat sealed or sewn to prevent fraying of the graft material 38. Stent 62 is secured to the flaps 58, 60, such as by sewing the stent 62 thereto in the locations where the stent 62 passes behind the flaps 58, 60. When deployed in an aorta 10, the stent 62 biases or pushes the graft material 38 forming the flaps against the aorta wall 12 as shown in FIG. 2, while the windows 76, 78 are positioned over the intersection of the aorta 10 and superior mesentery artery 30 and the celiac trunk 20 as also shown in FIG. 2. Preferably, as will be described further herein, stent 62 is formed as a portion of a stent frame 64, which includes therein stent 62 and a plurality of additional stents 80 (FIG. 7) such that the free circumference, i.e., the circumference prior to placement in the stent graft 32, of the stent 62 may be slightly smaller in circumference than that of stents 80, so as to prevent or reduce the possibility of overextension of the stent 60 into the aorta wall 12 where the portions of the stent 62 in the window(s) 76, 78 contacts the aorta wall 12. The flaps 58, 60 provide, when biased against the aorta wall 12, resistance to rotational and lateral movement of the stent graft 32 in the aorta 10.

Similarly to the arrangement shown in FIG. 3, FIGS. 3A and 3B show alternate embodiments of stent graft end windows, 76′ and 78′, respectively. In FIG. 3, the edge of the graft material for the window terminates and is bonded (by sewing or another suitable bond) to the stent graft wire that borders the window opening. The chance of the wire of the stent penetrating the surrounding tissue is reduced by distributing the wire load over a larger area by tying the edge of the stent wire to the surrounding graft material. In FIG. 3B, a pair of expansion limiting members 76″ and 78″ act as flexible tensile members to prevent the overexpansion of the first (top) end 41 of the stent graft 32. The expansion limiting members 76″ and 78″ can be made of fabric and bonded or tied to the graft material or it can be a wire section that is tied or bonded between or across the span between adjacent crowns of the end stent 62′ as shown in FIGS. 3A and 3B. During delivery, the expansion limiting member is compressed with the stent graft in the delivery sheath.

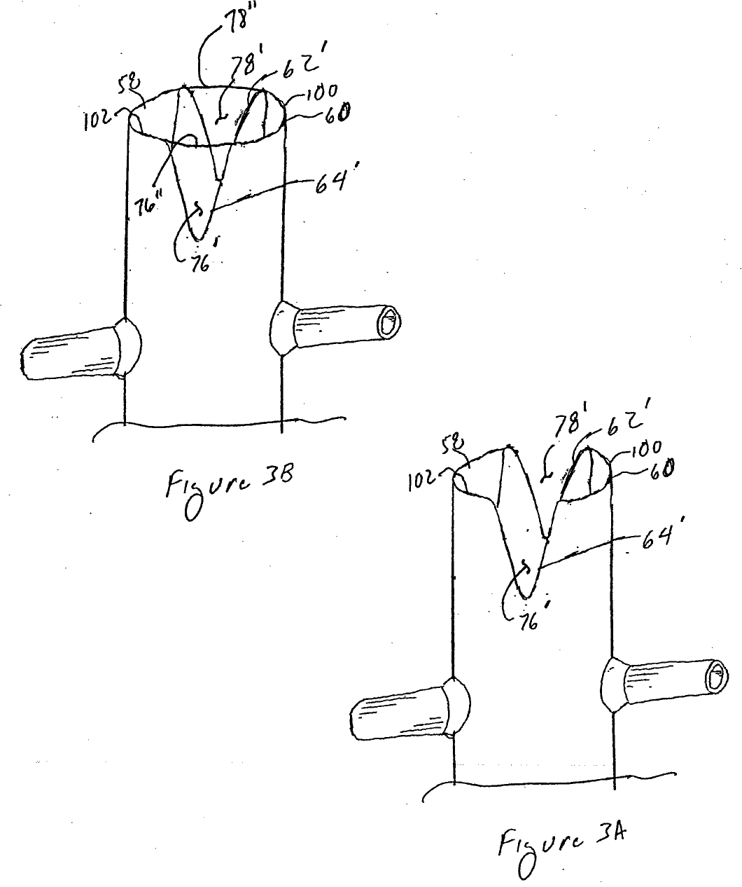

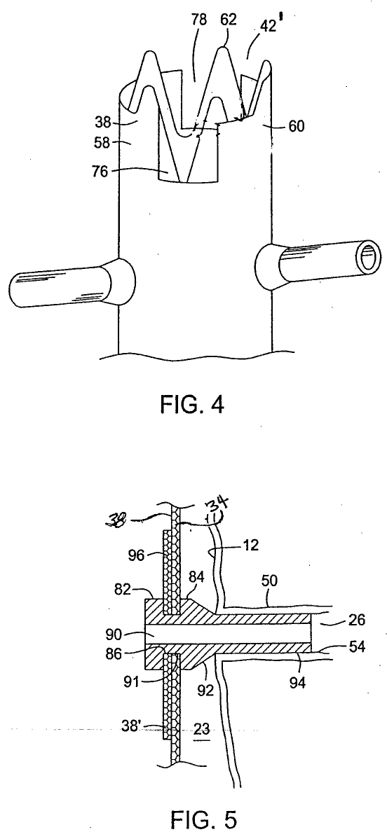

The first end 42 of the stent graft 32 is shown in FIGS. 2 and 3 as having, but for the windows 76, 78, generally continuous semi- or partially cylindrical ends 100, 102. At each of these open cylindrical ends 100, 102, the graft material 38 is folded back and sewn, heat sealed or otherwise secured against fraying. Referring to FIG. 4, an additional configuration of a first end 42′ is shown. In FIG. 4, the first end 42′ of the stent graft is scalloped, such that the graft material 38 of the flaps 58, 60 is cut inwardly of the first end 42 of the stent graft 32, such that the graft material 38 of the flaps 58, 60 follows, at least in part, the profile of the stent 62 above the edge of the graft edge shown in FIG. 3.

Referring now to FIGS. 2, 3 and 5, the renal extensions 50, 52 (only extension 50 shown in FIG. 5) are configured to engage into the renal arteries 24, 26 (only artery 26 shown in FIG. 5) and against the inner wall (54 or 56) thereof, and also be secured to the body 34 of the stent graft 32 with a flow conduit 90 extending therethrough. Each renal extension 50, 52 may have the construction of FIG. 5, wherein the extension is in the form of a grommet having an inner lip 82, an outer lip 84, a recess 86 disposed therebetween to be received and secured in an aperture 91 extending through the stent graft 32, and a tubular extending portion having a tapered portion 92 extending, when assembled, into a renal artery 24, 26 and in engagement against the wall thereof, and a generally circular cylindrical portion 94 extending therefrom for further projection into the renal artery and sealing against the wall 54 (or 56) thereof. Apertures 90 are generally circular holes in the sidewall of the stent graft 32, having a circumference slightly larger than that of the recess 88. To ensure secure retention of the renal extensions 40, 42 in the stent graft 30, a ring 96 ( or a plurality thereof) of additional graft material 38′ may be provided in the stent graft 30 at the aperture 90, to form a more rigid surface for the aperture than would occur with a single layer of graft material 38. Alternatively, rather than a ring 96 of additional graft material 38′, a metal or polymer ring may be sewn about the apertures 90 to provide a secure location for receipt of the renal extensions 50, 52.

The renal extensions 50, 52 are preferably configured by being molded of a biocompatible elastomer, such as silicone, such that they may be readily compressed for insertion into a sheath for delivery into an already deployed stent graft 32 at the aneurysmal location, yet have sufficient rigidity and elasticity to conform to a branch vessel and if needed, extend outwardly from the stent graft 32 through the gap 23 between the stent graft 32 body 34 and the adjacent aorta wall 12 at the renal arteries 24, 26. For example, where the aneurysmal sac 18 extends longitudinally such that the diameter of the aorta 10 at the renal arteries is distended, then the stent graft 32 might not contact the wall 12 of the aorta 10 adjacent to the renal arteries 24, 26, the extensions 50, 52 will bridge this gap 23 and also sealingly engage against the inner wall 54, 56 of the renal arteries 24, 26. The renal extensions 50, 52 also provide structural support for the stent graft 32 in the aorta, as they provide a stand off from the adjacent aorta wall 12 and provide resistance to torsional and lateral motion of the stent graft 32.

The stent graft 32 of this embodiment is a bifurcated stent graft, such that body 34 of the stent graft 30 includes, as shown in FIG. 2, a main body 120 having a major diameter trunk 122 portion and a minor diameter first leg 124 extending from one end of the trunk 122, and a second leg aperture 126. A second leg 128 is receivable within, and sealingly engageable against the inner surface of the second leg portion of the main body 122, thereby sealing the second leg aperture 126. To support the graft material 50 in an open tubular position when deployed into an aneurysmal descending aorta, stent frames 64, 64′ (Shown in FIG. 7) are provided. Stent frame 64 is received within main body 122 and stent frame 64′ is received in the second leg 128. In the embodiment shown, the stent graft 32 is intended to show the general configuration of a Talent AAA Bifurcated Stent graft sold by Medtronic AVE of Santa Rosa Calif., except the upper portion of the trunk 122 thereof is modified to accommodate the renal extensions 44, 44′ and is further extended to form and accommodate the windows 76, 78.

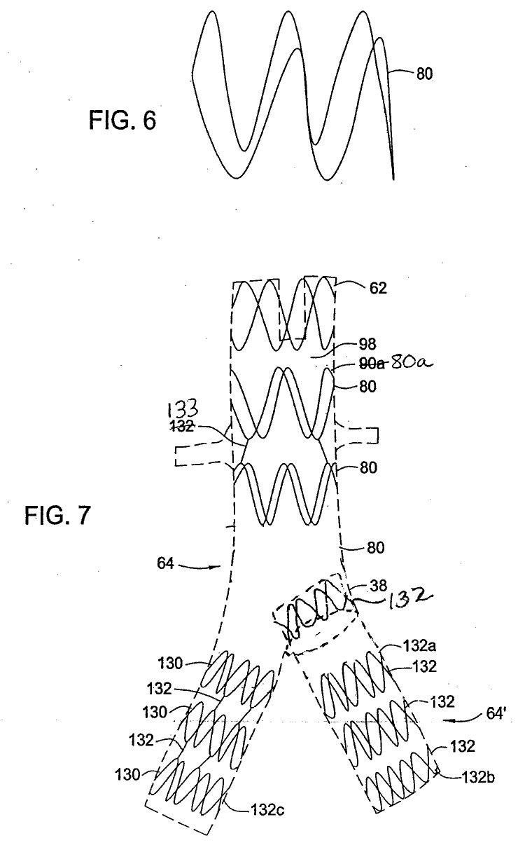

Referring now to FIGS. 6 and 7, each stent frame 64, 64′ is comprised of a plurality of stent elements. Referring firstly to stent frame 64, this stent frame includes stent 60, as well as a plurality of stents 80 (A single one of which is shown in FIG. 6 in perspective) of a common circumference slightly larger than that of stent 60, as well as a plurality, in this embodiment three smaller circumference leg stents 130. In this embodiment, the end stents of the set of three leg stents 130 are connected longitudinally by a connector bar 132, as are the end stents of the set of three stents 80. Also, as is shown in FIG. 7, the stent frame 80 is shown as it will be positioned in the graft material 38, which is shown in dashed line phantom in the Figure. The stents 60, 62 and 122 are sized and arranged to be received within the graft material 50, such that stents 62 and 122 will be slightly larger, in circumference, than the adjacent graft material into which they are deployed, such that they outwardly bias the graft material 38 to maintain the stent graft 32 as an open tubular structure. Additionally, stent 80a, as shown in FIG. 7, is located within the stent graft 32 at the sealing region 98 of the stent graft which will, when stent graft 32 is deployed, engage the aorta wall 12 at a location between the renal arteries 24, 26 position and that of the superior mesentery artery 30 and the celiac trunk 20. Thus, stent 80a is also used to bias the graft material 38 into sealing engagement with the adjacent aorta wall 12. Additionally, a plurality of leg stents 132 are separately provided and interconnected with spacing bars 132 to form stent frame 62′ for receipt within second leg 128. The uppermost leg stent 132 in second leg 128 is sized to ensure biasing of the second leg 126 graft material 38 into sealing engagement with the leg opening 126 in the main body 122, and the lowermost stents 130b, 130c in the legs 124, 126 are sized to bias the graft material of the legs 122, 126 adjacent their ends 45, 47 into sealing engagement with the adjacent aorta wall 12.

To form the main body 122 and second leg 128 of stent graft 32, the stents making up the stent framework 64, 64′ is compressed and inserted within or located to surround the envelope of the graft portions making up the main body 122 and second leg 128, and then allowed to expand. The stents of the stent framework 64, 64′ are then sewn to the adjacent graft material 50, to secure the stent frames 64, 64′ to the graft material 38 and form the components of the stent graft 32.

Referring now to FIG. 8, there is shown an alternative embodiment of the stent graft according to the present invention, wherein the upper end 42 of the stent graft 32 of FIGS. 2 through 7 is modified, and such that the windows 76, 78 which are formed by slots, are replaced with fully encircled windows 152, 154, as shown extending through the wall 156 of the first end 158 of a second embodiment of a stent graft 150. Each window is formed by cutting an elongate ovoid or elliptical hole through the graft material 164 at a position inward of the upper end 158 of the stent graft 150, and the edges of the holes are sewn, heat sealed or otherwise prepared to prevent fraying. A stent 160 of a stent frame (not shown) is received within the upper end 158 of the stent graft 150, such that the wire 162 forming the stent 160 may cross over the windows 152, 154. Stent 162, in contrast to stent 62 of the embodiment shown and described with reference to FIGS. 2 through 7, is preferably of the same circumference of the adjacent stents in the stent frame, as the stent 162 is fully restrained within the graft material 164 and thus will not overextend into the aorta wall 12 when deployed.

The material composing the graft material 38 or 164 of the stent grafts 32, 150 may be any biocompatible material that is mechanically stable in vivo, and is capable of preventing or substantially reducing the possibility of the passage or flow of blood or other body fluids there through. Typical materials for graft 24 include biocompatible plastics such as implantable quality woven polyester. Such polyester material may also include, therewith, components such as collagen, albumin, of an absorbable polymonomer or of a biocompatible fiber. Additionally, non-resorbable elastomers or polymers such as silicone, SBR, EPDM, butyl, polyisoprene, Nitril, Neoprene, nylon alloys and blends, poly(ethylene-vinyl-acetate) (EVA) copolymers, silicone rubber, polyamides (nylon 6,6), polyurethane, poly(ester urethanes), poly(ether urethanes), poly(ester-urea), polypropylene, polyethylene, polycarbonate, polytetrafluoroethelene, expanded polytetrafluoroethelene, polyethylene teraphthalate (Dacron) polypropylene and polyethylene copolymers.

The material from which the stents are formed is preferably a shape memory material, such as Nitinol, which, when compressed and cooled to a very low temperature such as by being sprayed with liquid nitrogen bursts, will maintain its compressed shape, but when heated back to room temperature will regain its original shape if unrestrained.

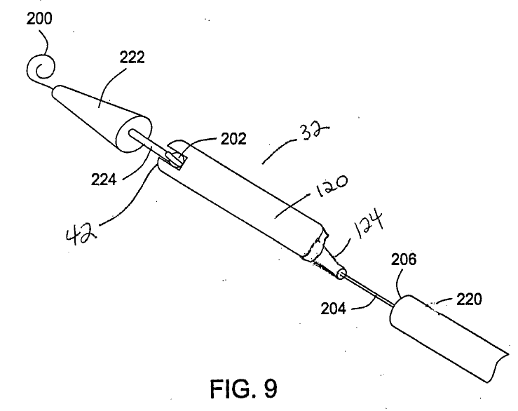

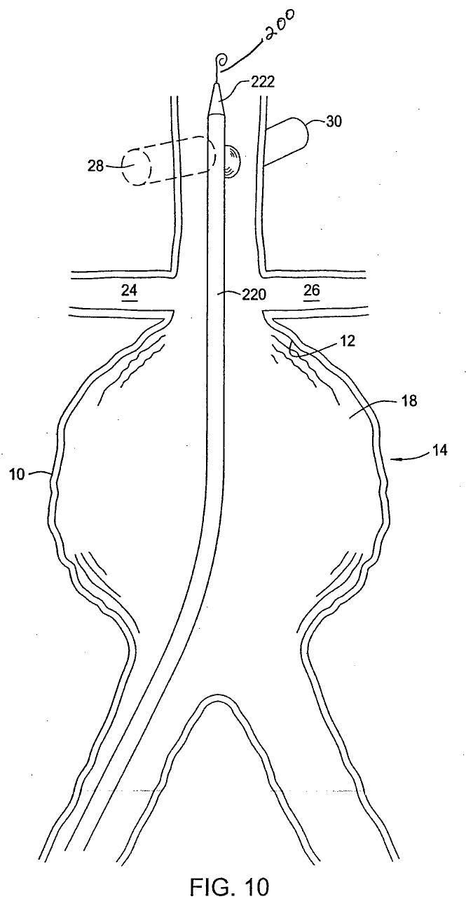

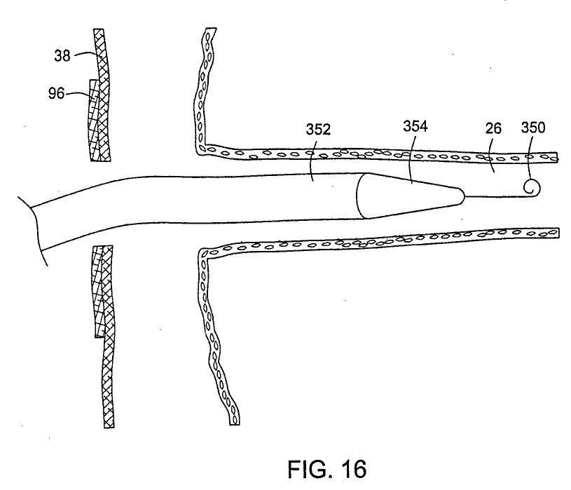

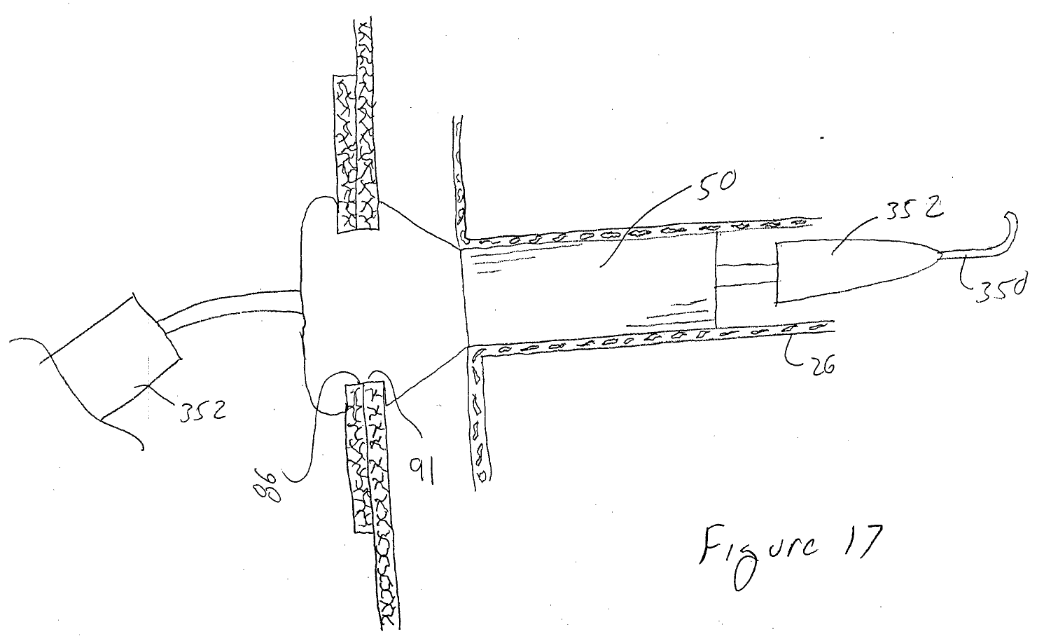

Referring now to FIGS. 9 through 17, the deployment of the stent graft 32 is shown, it being understood that the deployment procedure for stent graft 150 would include the same methodology. Referring initially to FIG. 9, in preparation for the stent graft insertion and deployment, a guidewire 200 is inserted into the patient's femoral artery and guided up to the upper portion of the aorta. Guidewire 200 may also include, alternatively, a balloon 202 and an inflation tube 204 for a balloon 202. The stent graft 32 main body 120 having been compressed, as shown in FIG. 10, possibly by using cooled bursts of liquid nitrogen and is compressed into a sheath, the compressed shape of FIG. 10. A similar operation is performed on second leg 128 and it is placed within its own sheath 161. Additionally, the renal extensions 50, 52 are also compressed and placed into catheter sheaths (Only sheath 352 shown in FIGS. 16 and 17)

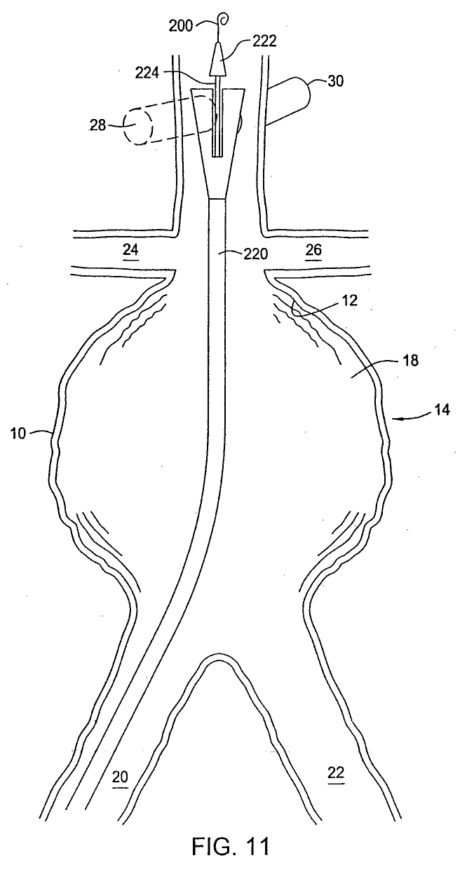

To deploy the stent graft 32, incisions are first made into the legs of the patient, such that the iliac arteries can be accessed at the leg location. The guidewire 200 is guided up the artery until it is positioned above (or beyond) the deployment location for the stent graft 32 as shown in FIG. 10. The catheter 220 having sheath 206 is introduced into the iliac artery in one leg. The catheter 220 is then moved along the guidewire 200, the catheter including a tapered introduction portion 222 which helps to pass the catheter through slightly restricted locations along the artery. The catheter 220 is introduced to the position shown in FIG. 11, above the deployment location for the stent graft. To locate the catheter 220, as well as the guide wire 200 and the stent graft 32 in proper deployment position, the aneurysmal region 14 of the aorta 10 is radiologically marked, and the stent graft 32 includes radiological marks thereon, such that the catheter 220, guidewire 200 and stent graft 32 may be fluoroscopically visualized by the practitioner deploying the stent graft.

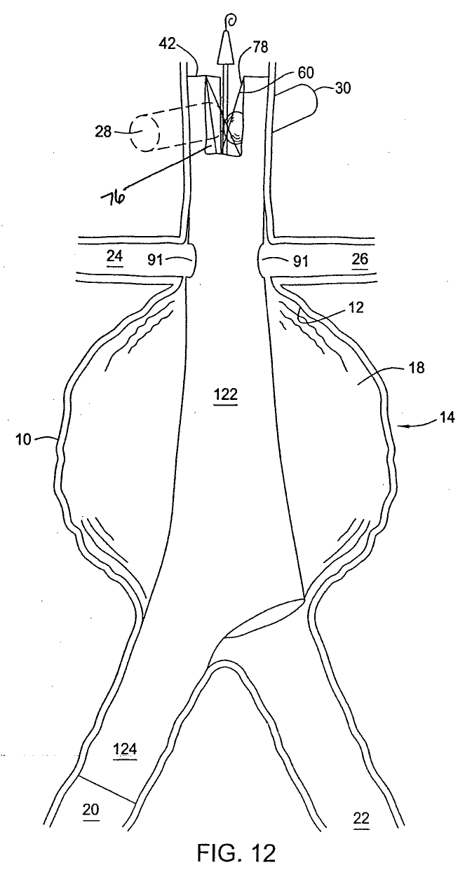

Once the catheter 220 is properly positioned for deployment as shown in FIG. 11, the sheath 206 is then retracted from the body through the insertion, while a stop (not shown) located within the sheath 206 maintains the graft in a stationary position against the main body 120 relative to the aorta 10. As the sheath retracts, the first end 42 of the stent graft 42 is released from the sheath 206, and the practitioner may rotate the catheter 220 and sheath 206 to enable alignment of the windows 76, 78 with the superior mesentery artery 30 and the celiac trunk 20 and the apertures 91 for the renal extensions 50, 52 in alignment with the renal arteries 24, 26. Radiological markers are provided on the stent graft 32 to enable the practitioner to assess longitudinal and rotational position of the stent graft main body 122 as the main body 122 is deployed. The sheath 206 is then fully retracted to release the first leg 124 within right iliac artery 20 as shown in FIG. 12. Once the stent graft delivery catheter is retracted, the balloon 204 on the guide wire may, if needed, be inflated to grip the interior of the stent graft and press out any wrinkles which may have occurred during deployment. Once the main body 122 is deployed and any wrinkles are removed with balloon 204, the balloon 204 and guide wire 200 are removed or retracted, as the catheter has already been removed or retracted.

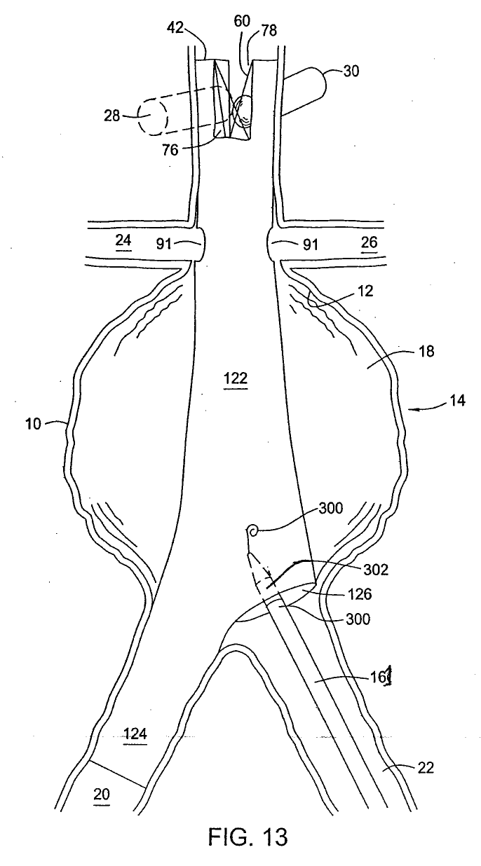

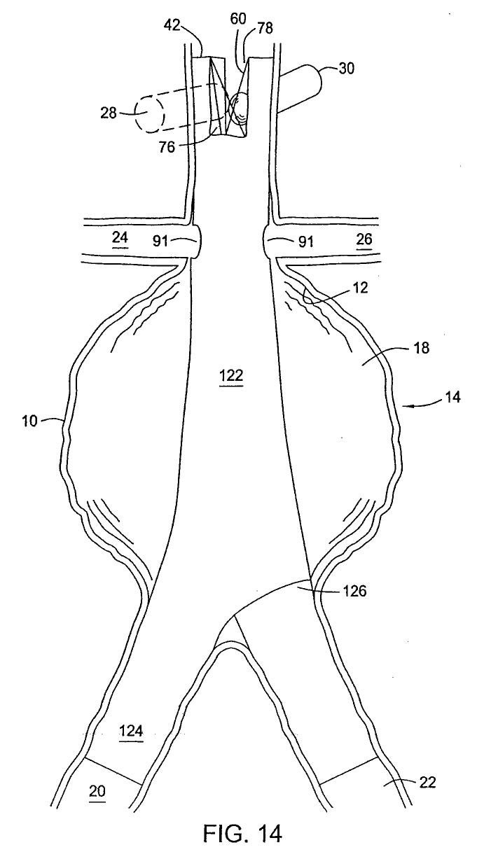

Once the main body portion 122 is deployed, the second leg 126 may be deployed. To do so, a guidewire 300 is introduced at a second leg incision location, and guided up the left iliac artery to a position within the main body portion 122, i.e., through the second leg aperture 126. Thence, as shown in FIG. 13, a second catheter 302 holding the second leg 128 is fed along the guidewire 300, until it is positioned within the main body portion 122 as shown in phantom in FIG. 13. Then as was performed with the main body portion 122, the introduction portion of the catheter 302 is extended away from the sheath 160 holding the second leg 128, and the second leg 128 is deployed such that one end thereof is positioned in the second leg aperture 126 and the second end thereof seals against the wall of the left iliac artery 22 as shown in FIG. 14. A balloon on the guide wire or a separate catheter (not shown) may be inflated and used to remove any wrinkles in the second leg 128. The catheter 302, along with guidewire 300, are then removed through the leg incision.

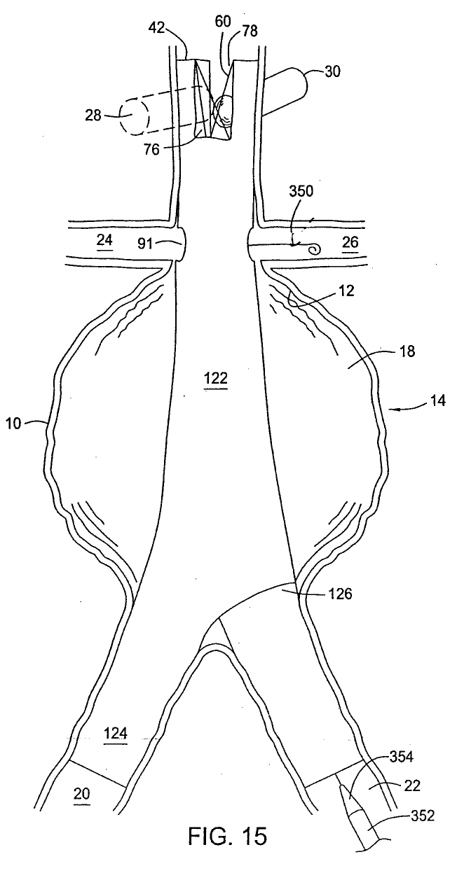

Referring still to FIG. 14, the stent graft 32 is deployed, but for the renal extensions 50, 52. Referring now to FIG. 15, the deployment of renal extension 50 is shown, it being understood that a similar procedure may be used to deploy renal extension 52. Initially, a guidewire 350 is fed through one of the leg incisions, and guided into the interior of the stent graft 32 main body portion 122 and thence outwardly through aperture 91 and thence into renal artery 26.

Once the guidewire 350 is located within the renal artery, a renal extension deployment catheter 352 is guided along the guidewire 325 to a position wherein the sheath holding the renal extension 50 extends into the renal artery and is positioned such that the introduction portion 352 thereof is located beyond the deployment location of the renal extension 50. Thence, the introduction portion 352 is moved inwardly of the renal artery 26, exposing the open end of the sheath. Thence, as with the deployment of the main body portion 122 and the second leg 128, the sheath is withdrawn while the renal extension 50 is stationary, resulting in the renal extension 50 deploying from the sheath. To properly position the renal extension 50, radiological markers may be employed, such that the distance between the marker and the recess 86 which fits over the aperture 91 in the graft material 38 is known to the practitioner, so that the renal extension 50 is deployed with the recess 86 directly within the circumference of the aperture 91. Additionally, a balloon (not shown) may be provided within the renal extension 50, such that if the renal extension remains in a collapsed state upon deployment, the practitioner may align the may align the renal extension 50 with the aperture 91, and inflate the balloon to its fully expanded free state. Once deployed, the renal extension 50 (as with renal extension 52), seals the aperture 91 and also seal against the inner wall of renal artery 26, to provide a flow conduit for blood while excluding blood flow to the aneurysmal sac 18.

What is claimed is:

1. An exclusion device for excluding fluid contact to an abnormality in a body flow lumen, comprising:
a body portion having a wall portion and at least opposed first and second open ends;
a first and a second slot, extending through the wall portion of said body portion, adjacent said first open end, each of said slots being positioned in circumferential opposition to one another and separated from each other by portions of the wall portion extending to said first open ends;
a first biasing member received in said body portion and configured to bias said wall portion of said body portion in a radially outward direction;
a second biasing member maintained within said body portion adjacent said first open end and configured to bias said wall portion radially outward to the same, or less, extent as that provided by the first biasing member.

2. The exclusion device of claim 1, wherein:
said first biasing member is a stent frame.

3. The exclusion device of claim 1, wherein said second biasing member is a stent.

4. The exclusion device of claim 3, wherein said first biasing member is a stent frame, and said stent is of a smaller free diameter than said stent frame.

5. The exclusion device of claim 3, wherein said stent is a wire hoop, and said wire hoop includes a plurality of apexes separated by relatively straight lengths of wire to form a repeating pattern about the circumference of the stent; and
the slots have a geometry different than that of the repeating pattern on the stent.

6. The exclusion device of claim 5, wherein said slots follow the profile of one of said repeating patterns, and a secondary restraining member extends across the slots and is secured to the flaps at either side of the slots.

7. The exclusion device of claim 6, further including a plurality of scallops of a size different than that of said slots, extending along said wall portion inwardly of said first end.

8. The exclusion device of claim 7, wherein said wall portion is provided as a graft material.

9. The exclusion device of claim 1, wherein said exclusion device is a bifurcated stent graft.

10. The exclusion device of claim 1, wherein said slots are sized and arranged to be positioned, when said exclusion device is deployed, in alignment with branch artery locations on the flow lumen into which the exclusion device will be deployed.

11. The exclusion device of claim 1, wherein said slots include a perimeter entirely bounded by said wall portion.

12. The exclusion device of claim 6, wherein said secondary restraining member is formed of a continuous hoop of said wall portion.

13. A stent graft for deployment into an aneurysmal artery location, comprising:
a tubular graft material having at least a first open end and a second open end;
a biasing member receivable adjacent said first open end and a stent framework received in contact with said tubular graft material in a location between said biasing member and said second open end;
at least one slot, extending in said graft material from said first open end and terminating to the first open end side of said stent frame; and
wherein said biasing member and said stent outwardly bias said graft material, and the bias provided by said biasing member is less than or equal to that provided by the stent framework on the graft material.

14. The stent graft of claim 13, wherein said biasing member includes a stent.

15. The stent graft of claim 14, wherein said stent has a smaller free diameter than said stent frame.

16. The stent graft of claim 14, wherein said biasing member further includes a restraining member extending across said slot.

17. The stent graft of claim 16, wherein said restraining member extends circumferentially about said first end of said tubular body.

18. The stent graft of claim 12, further including a second slot, of the same configuration as said at least one slot, located diametrically opposed to said at least one slot and extending inwardly of said first end in said graft material.

19. The stent graft of claim 18, further including a restraining member extending about the circumference of said first open end, and spanning said slots.

20. The stent graft of claim 19, wherein said slots are separated, along the circumference of said tubular body, by flaps extending to said open end of said stent graft.

21. The stent graft of claim 20, wherein said slots are rectangular.

22. The stent graft of claim 20, further including at least one scallop extending inwardly of said wall portion at said first end and into said flap.

23. An exclusion device for excluding fluid contact to an abnormality in a body flow lumen, comprising:
a body portion having a wall portion and at least opposed first and second open ends;
a first and a second opening, extending through the wall portion of said body portion, adjacent said first open end, each of said openings being positioned in circumferential opposition to one another and separated from each other by portions of the wall portion extending to said first open ends; and
a biasing member received in said body portion and configured to bias said wall portion of said body portion in a radially outward direction, said exclusion device spanning the location of said openings.

24. The exclusion device of claim 23, wherein said openings are circumferentially opposed slots extending along said wall portion from said first open end, and
said biasing member is configured to bias said wall portion, adjacent to said slots, differently than said wall portion at other locations on said exclusion device.
